(19) United States
(12) Patent Application Publication
(10) Pub. No.: US 2008/0227855 A1
(43) Pub. Date: Sep. 18, 2008

(54) Solid Dispersion of Hydrophobic Bioactive

(76) Inventors: Graham Buckton, London (GB); Stephen Brocchini, London (GB); John Fletcher, London (GB); Hisham Al-Obaidi, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/815,619

(22) PCT Filed: Feb. 10, 2006
(86) PCT No.: PCT/GB2006/000467

§ 371 Date: May 7, 2008

(30) Foreign Application Priority Data
Feb. 10, 2005 (GB) 0502790.9

(51) Int. Cl. A61K 31/343

(52) U.S. Cl. 514/462

(57) ABSTRACT

A stable composition of an amorphous component (such as a bioactive) and a carrier polymer is formed by mixing a bridging polymer with the bioactive and the carrier polymer, wherein the bridging polymer is a hydrogen bond donor to both the bioactive and the carrier polymer, thereby forming a composition in which the bioactive and the carrier polymer have less of a tendency to crystallise than if the bridging polymer were not present.

The present invention relates in general to a method of forming a stable composition of an amorphous component, and in particular to a method of producing microparticles of a bioactive in which the bioactive is stabilised in an amorphous form.

The pharmaceutical industry invests an enormous amount of money each year developing bioactives (drugs) for treatment of human and animal medical conditions. However, the development of a drug which has the required pharmaceutical effect on the body is not the end of the matter—that drug must also be formulated so that it can actually be delivered to the body to have the required effect.

This can be problematic when, for example, the drug has a slow dissolution rate, perhaps limited by low aqueous solubility. One reason for limited dissolution can be difficulties in the release of molecules of drug from a strong crystal lattice. It is often advantageous that certain drugs be stored in an amorphous form (no crystal lattice), because this tends to result in easier distribution of drug molecules into solution, giving a higher drug dissolution rate.

One technique for addressing this problem is to formulate a poorly water soluble drug as a solid dispersion. The term solid dispersion has been described as “the dispersion of one or more active ingredients in an inert carrier matrix at solid-state” (Chiou and Riegelman, (1971) J. Pharm. Sci. 60, 1281-1302).

The concept of using a solid dispersion to increase solubility is not a new one. Polymers such as polyvinyl pyrrolidone (PVP) have been used to stabilise the amorphous form of drugs. One reason why a drug may remain in an amorphous form in a polymeric system is the restricted mobility of drug molecules. The drug molecules would wish to realign and crystallise, but this process could be rather slow due to restricted diffusion in the polymer. It is thought that the glass transition temperature of the polymer will influence this, with high Tg values resulting in a lower tendency for drug molecule mobility. However, there are clear examples of drugs dispersed in polymers with high Tg in which there is relatively rapid crystallisation for example griseofulvin crystallisation from PVP (Shefter and Cheng, 1980 Int. J. Pharm. 6, 179-182).

Another reason why drugs may not crystallise in polymeric dispersions is if there is a favourable interaction between the drug and the carrier. For example Indomethacin has been shown to interact with PVP via hydrogen bonding between the carboxylic acid group of indomethacin and the carbonyl group of PVP providing a level of amorphous stability above that expected on the basis of glass transition temperature alone (Taylor and Zografi, 1997).

GB 1 504 553 (Sandoz Ltd) discloses incorporating the bioactive griseofulvin as a solid dispersion into a water soluble carrier, polyethylene glycol (PEG), in order to increase the dissolution rate of the griseofulvin. The process of tableting the composition substantially reduces the dissolution rate of the bioactive, and so cross-linked polyvinylpyrrolidone is incorporated to act as a disintegrating agent by reducing the cohesive forces between the griseofulvin and the PEG.

WO 01/034119 (Abbott Laboratories) discloses a pharmaceutical composition comprising a solid dispersion of a pharmaceutical compound in a water-soluble carrier (such as PEG), and a crystallisation inhibitor such as PVP or hydroxypropylmethylcellulose (HPMC).

EP 0 232 155 (Elan Corporation plc) relates to a controlled release formulation comprising an adsorbate of a mixture of a bioactive and an inactive substance (such as PEG, PVP or a methacrylate) adsorbed on a cross-linked polymer (for example a methylcellulose).

JP 55129220 A (Yamanouchi Pharma Co. Ltd) discloses a formulation of a bioactive such as griseofulvin compounded with either (a) a composition containing PVP, methylcellulose, hydroxypropyl cellulose and/or hydroxypropyl methylcellulose or (b) a mixture of (i) a composition containing one or more of PVP, urea, citric acid or mannitol and (ii) one or more of a surfactant, PEG, propylene glycol or glycerine.

Broman et al. (2001) Int. J. Pharm. 222, 139-151 discloses a number of solid dispersions prepared with the extremely poorly water soluble drug probucol and the water soluble polymers PVP, polyacrylic acid (PAA) or polyethylene oxide (PEO) and blends of these polymers. The physical state of the drug was observed to be dependent on the polymeric excipient. PVP and probucol contained probucol in its amorphous form, which was postulated to be due to hydrogen bonding (H-bond) interaction between the drug and the polymer. By contrast, the drug and PAA and PEO respectively contained probucol in its crystalline polymorph II form. Finally, tertiary mixtures of probucol/PVP/PAA and probucol/PVP/PEO are disclosed in which the drug is initially in its amorphous form but displays very long dissolution times resulting in no advantage for this system.

In accordance with a first aspect of the present invention, there is provided a method of forming a stable composition of a bioactive, comprising the steps of:
(i) providing a bioactive and a carrier wherein, if they alone are mixed, at least one of them has a tendency to crystallize,
(ii) mixing a bridging component with the bioactive and the carrier, wherein the bridging component forms hydrogen bonds with both the bioactive and the carrier, thereby forming a composition in which at least the bioactive has less of a tendency to crystallise than if the bridging component were not present,
   wherein preferably the bridging component is a hydrogen bond donor to both the bioactive and the carrier.

The onset of crystallisation is determined by the arrival of distinct peaks on the powder X-ray diffraction pattern. By contrast, the powder X-ray diffraction pattern for the amorphous state shows a “halo” effect without distinct peaks. A “tendency to crystallize” means that the bioactive and carrier exhibit the onset of crystallisation after a few days. The worst examples are so crystallised as to be unusable in a matter of weeks. By contrast, the compositions of the present invention are stable for months, and may be stable for years (preferably at least two years) if kept in dry conditions and at ambient temperatures, thereby giving them a viable shelf life.

The carrier and the bridging component are both preferably polymers, and most preferably polymers which are not cross-linked. The carrier polymer is preferably not polyethylene glycol.

In a preferred embodiment the bioactive has a tendency to crystallise but the carrier does not have a tendency to crystallise when the two components are mixed without the bridging polymer. In an alternative embodiment, the bridging polymer reduces the tendency to crystallise of both the bioactive and the carrier polymer.

By “interaction” between bioactive and carrier polymer or bridging polymer is meant any interaction which prevents the bioactive from seeding. This is because seeding (or clustering) increases the tendency of the bioactive to crystallise. Such interactions include but are not limited to van der Waals bonding, electrostatic interactions, hydrophobic interactions and (preferably) hydrogen bonding.

In a preferred embodiment, the bridging polymer is a hydrogen bond donor to both the bioactive and the carrier polymer. However, an alternative formulation comprises a bridging polymer which is a hydrogen bond acceptor with respect to a hydrogen bond donating drug and a hydrogen bond donating carrier polymer which would not otherwise interact and would not therefore form a stable composition in which the drug remained in its amorphous form.

Griseofulvin (a well known antifungal agent) is a poorly water soluble drug that has been shown to crystallise when dispersed in PVP (Shefter and Cheng, 1980 Int. J. Pharm. 6, 179-182). In accordance with the present invention, a more stable composition can be prepared of griseofulvin (the bioactive), PVP (the carrier polymer) and polyhydroxypropylmethacrylate (the bridging polymer). It is believed that the polyhydroxypropylmethacrylate donates hydrogen bonds to both the drug and the PVP, thereby acting as a bridge between them and stabilising the drug in its amorphous form.

A hydrogen bond is a non-covalent interaction that occurs amongst molecules. As a non-covalent interaction it is a relatively strong interaction that results from the interactions of relatively electronegative heteroatoms (such as oxygen and nitrogen) that are covalently bonded to hydrogen. The bond between the hydrogen and its heteroatom is partially polarised by the electronegativity of the heteroatom, resulting in a partial positive charge on the hydrogen atom. Molecules can be characterised as hydrogen bond donating molecules or hydrogen bond accepting molecules. Certain molecules have both characteristics, the most notable being water.

The electronegative heteroatom to which the hydrogen atom is attached (and by extension the molecule carrying that heteroatom) is known as the “hydrogen bond donor” since it is bonded to a polarised hydrogen atom. An electronegative atom (e.g. oxygen or nitrogen) not bonded to hydrogen (and by extension the molecule carrying that heteroatom) can interact with a hydrogen bond donor and is thus known as a “hydrogen bond acceptor”. In the case of water, the oxygen atom both “accepts” hydrogen bonds through its lone pairs of electrons and “donates” hydrogen bonds through its covalent bond to hydrogen.

In some instances it may be difficult to tell which heteroatom is which (if for example the hydrogen bond is predominantly covalent and shared). The convention is therefore to imagine the two heteroatoms being separated, and the hydrogen nucleus being retained by one of the heteroatoms. If this retention causes no increase in the heteroatom's positive charge, then that heteroatom is the donor. If however the heteroatom would become more positive by retaining the hydrogen, it is the acceptor. This convention is adopted herein.

Clearly, a particular polymer may be a hydrogen bond donor to one molecule and an acceptor for another, as the position is determined by relative electronegativity (indeed, some large molecules such as proteins can form intra-molecular hydrogen bonds, which can have a significant effect on molecular configuration). If the shorthand “H-bond donor” or “H-bond acceptor” is used herein in relation to a polymer therefore, it should be understood to mean donor/acceptor relative to the molecule with which the polymer forms hydrogen bonds.

Without wishing to be constrained by theory, the present applicant believes that, in the tertiary mixture of PVP/probucol/PAA disclosed in the Broman reference, there are hydrogen bond interactions between the probucol and the PVP (with the probucol acting as an H-bond donor), between the PVP and the PAA (with the PAA acting as an H-bond donor), and between the probucol and the PAA (with the probucol acting as an H-bond donor). There is no suggestion in the Broman reference of employing a polymer as a bridge between a carrier polymer and a bioactive that do not themselves interact.

In practice, the components of the inventive composition are combined by dissolving them in a solvent which is then evaporated once the components are sufficiently mixed or by mechanical activation (i.e. energetic milling of the materials). When using solvent, the solvent may be a single solvent (such as acetone) or a combination of more than one solvent (such as acetone and water).

Again when using solvents it is greatly preferred that the solid dispersion be fabricated by a process that rapidly causes evaporation of the solvent, such as by spray drying. Any suitable evaporation technique may be used in which the surface area of the material to be dried is substantially increased prior to evaporation. In the case of spray drying, the material is aerosolised in order to increase its surface area. As the droplets leave the aerosol nozzle, they are heated and the solvent evaporates from them extremely quickly.

In a preferred embodiment, the carrier polymer and the bridging polymer are substantially miscible. Miscibility can be determined by thermal analysis.

The present applicant has also surprisingly discovered that the order in which the components of the composition are mixed affects the stability of the resulting composition. However, the preferred mixing order for stability may not necessarily be the same as that which gives fastest dissolution. In a preferred embodiment therefore, the composition is prepared by first mixing the carrier and the bridging component and then admixing the bioactive. It should be emphasised that a more stable amorphous composition, compared to the situation in the absence of a bridging polymer, can be prepared no matter in what order the components are mixed, but that greater stability may be achieved by tailoring the order for a particular composition.

In a preferred embodiment, the proportion of the final composition that is bioactive is from 40 to 60% w/w. The remainder of the composition is preferably carrier polymer and bridging polymer (although there may be some residual solvent) and these are preferably from 0.05:1 to 3:1, more preferably from 0.5:1 to 3:1 and most preferably 0.5:1 to 1.5:1.

In accordance with a second aspect of the present invention, there is provided a method of forming a stable composition of an amorphous component, comprising the steps of:
(i) providing a bioactive and a carrier polymer wherein, if they alone are mixed, there is negligible interaction between them, so that at least one of them has a tendency to crystallize,
(ii) mixing a bridging polymer with the bioactive and the carrier polymer, wherein the bridging polymer interacts with both the bioactive and the carrier polymer, thereby forming a composition in which the bioactive and the carrier polymer have less of a tendency to crystallise than if the bridging polymer were not present. Preferably, the bridging polymer is a hydrogen bond donor to both the bioactive and the carrier polymer.

In a third aspect of the present invention, there is provided a composition obtainable by a method as defined above.

Without wishing to be constrained by theory, it is thought that the present invention differs from the prior art as follows:

### Sandoz Ltd. (GB1504553 A)

This reference relates to a specific situation wherein the bioactive griseofulvin is incorporated as a solid dispersion into a water soluble carrier, polyethylene glycol (PEG), in order to increase the dissolution rate if the Griseofulvin. Since the process of tableting substantially reduces the dissolution rate of the drug, cross-linked polyvinylpyrrolidone (PVP) is incorporated to act as a disintegrating agent by reducing the cohesive forces between the drug and the PEG. In contrast, the present invention relates to a more general formulation problem, that of enabling solid dispersions to exist. The situation is opposite to that envisaged in the Sandoz reference in that instead of trying to decrease the molecular interactions between the bioactive and the carrier polymer, the present method seeks to increase these interactions in order to increase the stability of the amorphous form of the bioactive. It does this by introducing a bridging polymer which simultaneously interacts via hydrogen bonding, with both the bioactive and the carrier molecules. By this means, the carrier is able to reduce the tendency of the molecules of bioactive to undergo rearrangement to form a crystal lattice.

### Abbott Laboratories (WO01/34119 A)

This relates to solid dispersion formulations of bioactive in a water-soluble carrier such as PEG together with a crystallisation inhibitor such as polyvinylpyrollidone. The present differs in that it seeks to address situations where PVP alone is not able to act effectively as a crystallisation inhibitor. As discussed above, it does this by providing a bridging polymer to enable the PVP to interact indirectly with the bioactive, thus reducing its tendency to crystallise.

Furthermore in this instance the carrier (PEG) is likely to crystallise on storage, that being one of the problems with PEG based dispersions. Hence PVP is most probably added here to limit crystallisation of the PEG carrier. The present method is employed in cases in which PVP (for example) would fail to inhibit the crystallisation of the drug substance (due to limited H-bonding) and a second polymer is added to link PVP to the drug. This reference does not work in that way; in fact PEG and PVP are similar in terms of their H bonding and hence would not complement each other in the way required by the present invention.

### Elan Corp. (EP0232155 A2)

This relates to a controlled release formulation wherein the bioactive together with an “inactive substance” is incorporated via solvent evaporation into a matrix comprising a cross linked polymer. The “inactive substance” is selected for its ability (due to its solubility properties) to control the rate of dissolution of the bioactive, when the formulation is ultimately dispersed in water. For example, water-soluble “inactive substance” will speed up the rate of dissolution of the bioactive from the matrix, whereas a water insoluble “inactive substance” will slow it down, resulting in delayed release. In contrast, the present method does not seek to control the rate of dissolution of the bioactive when the formulation is ultimately dispersed in water. Instead, it aims to increase the interaction of the bioactive with the polymer matrix during the formulation stage, such that the bioactive does not crystallise during storage. By maintaining it in an amorphous state, it will then undergo more rapid dissolution when eventually dispersed in water.

Note that the Elan reference specifically refers to the use of a cross-linked polymer whereas cross-linked polymers are not used in the present method.

### Yamanouchi Pharmaceutical Co. (JP55129220 A)

This relates to formulations of poorly soluble medicinal components together with a base component such as PVP, and possibly further component(s) such as PEG, in order to increase the rate of action and bioavailability of the medicinal component. However there is no suggestion in the abstract that the further components are incorporated in order to increase the interaction between the bioactive and the base component. In fact the glycols listed would have the same hydrogen bonding potential as the PVP carrier and hence would not stabilise any better than PVP alone.

E. Broman et al, Int. J. Pharmaceutics, 222, 139-51, (2001)

This publication describes the preparation of solid dispersions of a poorly water-soluble drug, probucol together with different water-soluble polymers, viz. PVP, PAA and PEO. The polymers were either used individually, or else as mixtures of two, one being PVP. The drug was found to be amorphous in the PVP formulation and crystalline in the both PAA and PEO. The situation with PVP is in contrast to that in GB0502790.9, where the bioactive is largely crystalline in PVP and requires a bridging molecule to render it amorphous.

When formulated in PVP together with one of the other two polymers, the probucol was generally amorphous. However it was found that this had little effect on the release behaviour of the drug during dissolution, which is the main measure of success of this formulation approach. Thus no benefits were seen compared with using single polymers. This is in contrast to the present method, where incorporation of a bridging molecule alleviates the problem of crystallisation within a single polymer matrix.

A number of preferred embodiments of the invention will now be described with reference to the accompanying drawings, in which:

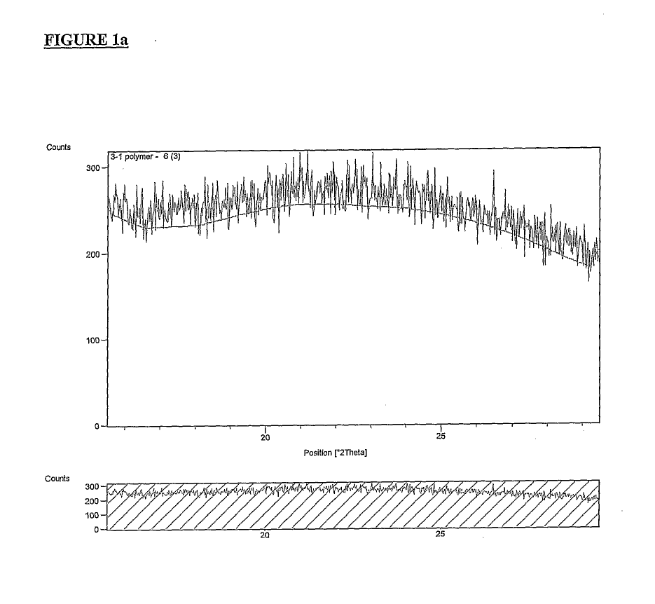

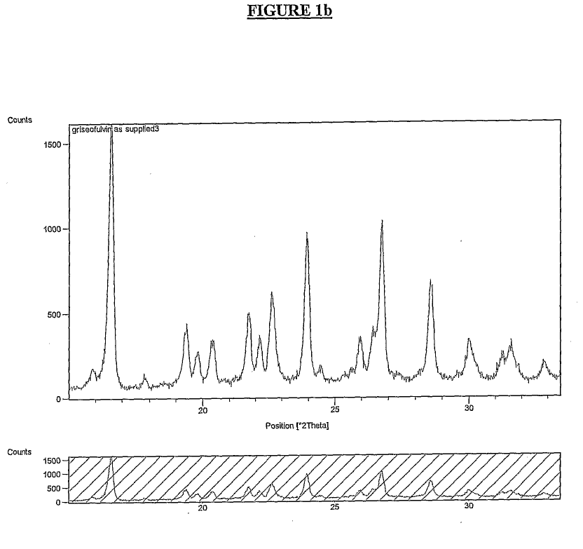

FIG. 1a is an XRPD scan of a composition in accordance with the invention.

FIG. 1b is an XRPD scan of pure crystalline griseofulvin, part of a set used to calculate the level of crystalline material in the invention

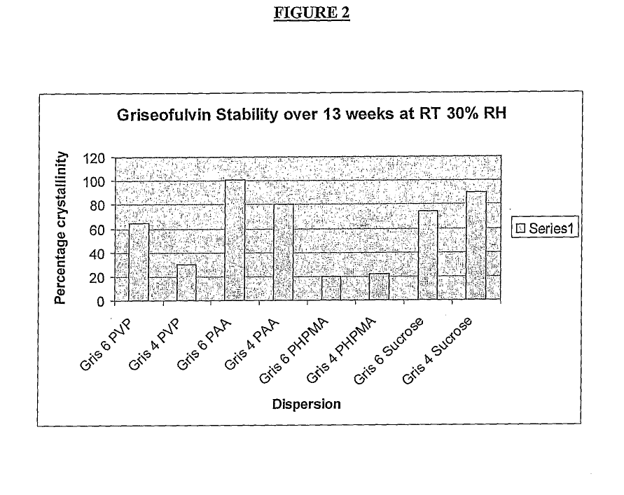

FIGS. 2 to 5 are graphs showing the level of crystalline material present in various compositions according to the invention after 13 weeks of storage under various storage conditions.

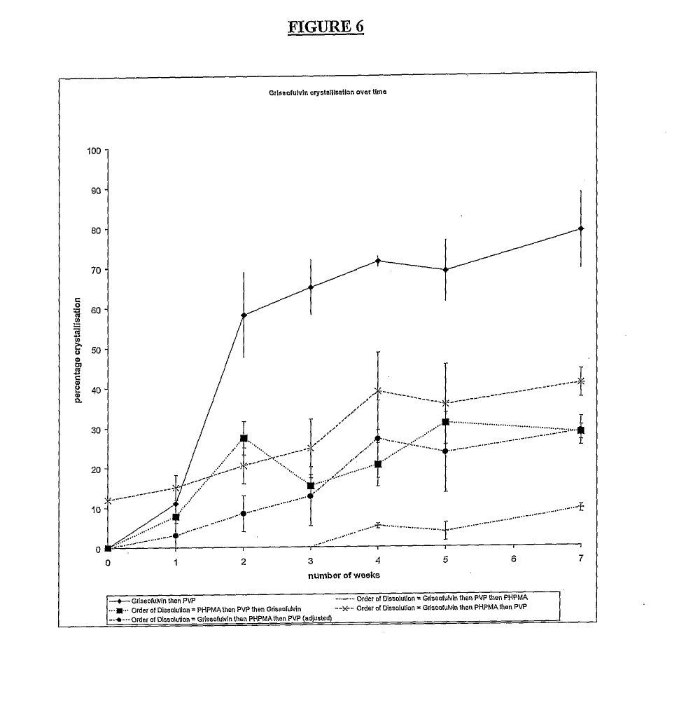

FIG. 6 shows the level of crystallinity of a number of different compositions over time, with the difference between the compositions being the order in which the components were added.

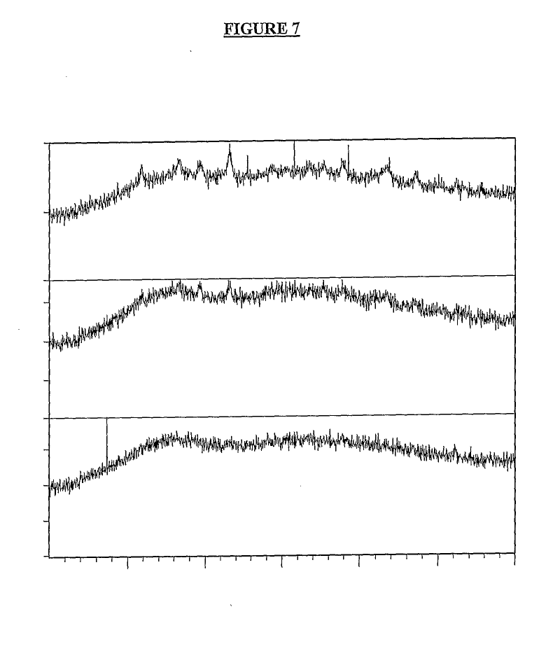

FIG. 7 shows the The x-ray powder diffraction pattern of solid dispersion of

(a) griseofulvin:PHPMA:PVP (3:1:1) solid dispersion,
(b) griseofulvin:PVP:PHPMA (3:1:1) solid dispersion and
(c) PVP:PHPMA:griseofulvin (1:1:3) solid dispersion stored at 84% RH at room temperature after 12 weeks from day of preparation; the top trace has signs of crystallinity whereas the bottom does not.

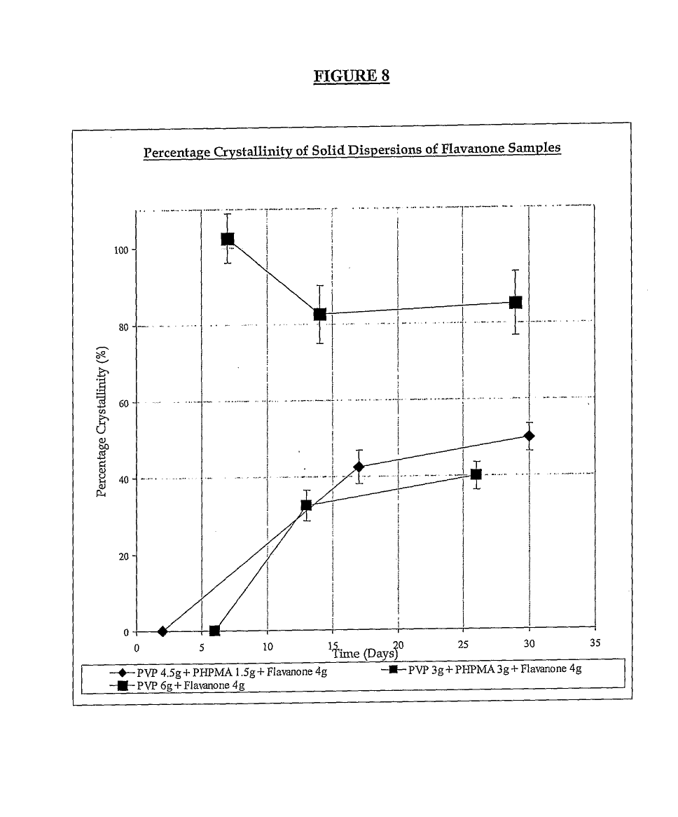

FIG. 8 shows the level of crystallinity of a number of different compositions over time, with the difference between the compositions being the presence or absence of PHPMA.

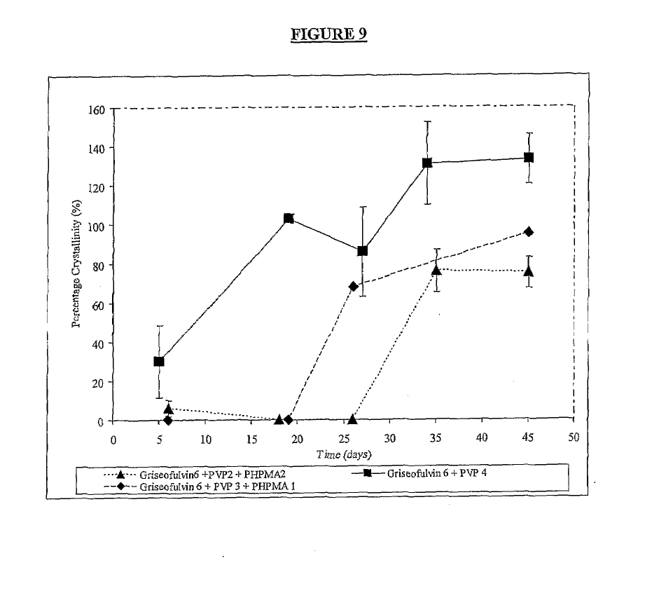

FIG. 9 shows the level of crystallinity of a number of different compositions over time, with the difference between the compositions being the ratio of PVP to PHPMA.

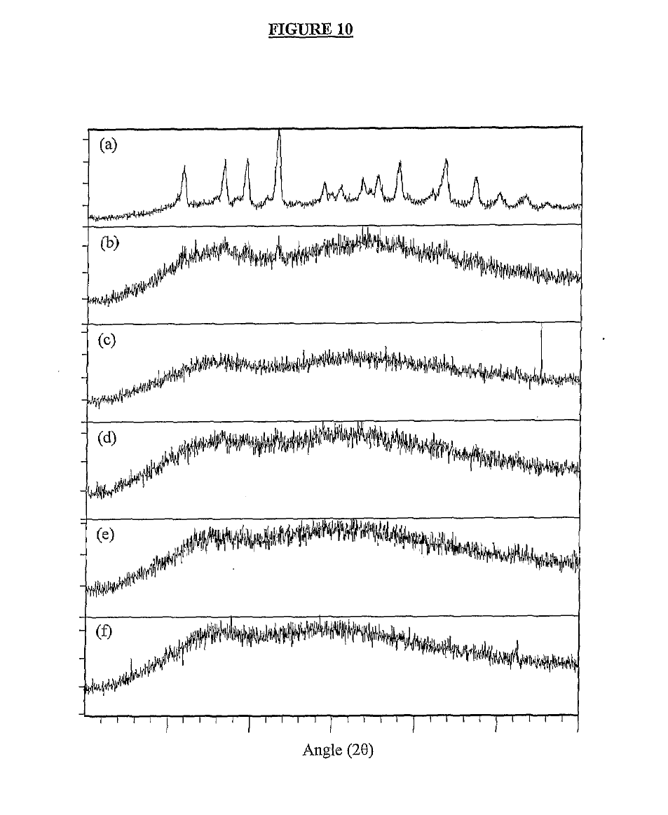

FIG. 10 shows the x-ray powder diffraction pattern of solid dispersion of (a) griseofulvin:PHPMA:PVP (2.5:0.25:2.25, (b) griseofulvin:PHPMA:PVP (2.5:0.5:2), (c) griseofulvin:PHPMA:PVP (2.5:0.75:1.75), (d) griseofulvin:PHPMA:PVP (2.5:1:1.5), (e) griseofulvin:PHPMA:PVP (2.5:1.25:1.25) and (f) griseofulvin:PHPMA:PVP (2.5:1.5:1) stored at 84% RH at room temperature after 12 weeks from day of preparation.

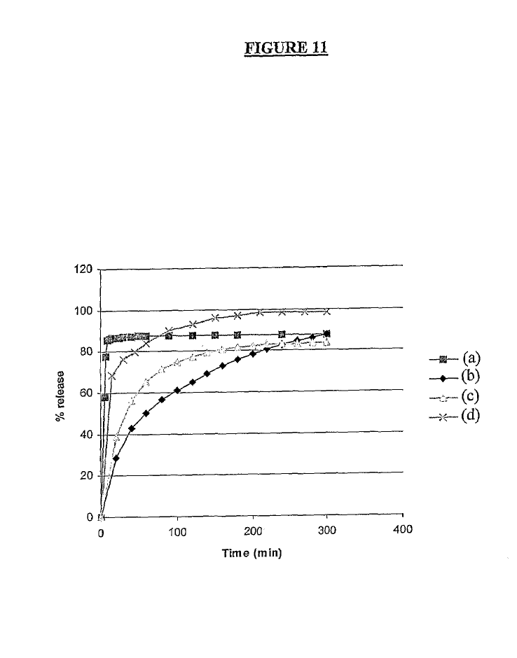

FIG. 11 shows the dissolution profile of (a) griseofulvin:PHPMA:PVP (2.5:1.25:1.25) solid dispersion in phosphate buffer (pH 6.5) and 0.2% SDS buffer (pH 6.8) at 37 C.° at 100 rpm, (c) crystalline griseofulvin in phosphate buffer (pH 6.8) at 37 C.° at 100 rpm and (d) griseofulvin:PVP (2.5:2.5) solid dispersion in phosphate buffer (pH 6.8) at 37 C.° at 100 rpm.

### EXAMPLES

### Methodology

All dispersions were spray dried using a Niro SD spray dryer connected to a nitrogen generator.

Solutions for dispersions were prepared by dissolving griseofulvin, a major excipient (PVP) and a minor excipient in an acetone water co-solvent system. To ensure homogeneity, the dispersions were allowed to mix for eight hours before the dispersion was spray dried using a Niro SD system under nitrogen conditions with an inlet temperature of 65° C. and an outlet temperature of 45° C. The feed rate was around 15% of maximum and the nitrogen flow rates were 20 kg/hr and 2 kg/hr for the drying and atomization rates respectively.

The samples were then stored under constant vacuum for 24 hours to allow for the removal of solvent before being transferred to the required conditions.

A Philips PW37010 X-ray Powder Diffractometer (Philips, Cambridge, UK) was used to analyse the samples for crystallization, with a sample XRPD scans shown in FIGS. 1a and 1b.

To prepare the sample for analysis, about 200 mg of sample was loaded into a circular well in the sample holder which had a diameter of approximately 260 mm and a depth of approximately 1 mm.

This was compressed to produce a flat surface before being loaded into the XRPD. The samples were then scanned using an X-ray lamp tension of 45 kV and 30 mA. A continuous scan was performed between 15.5° 2Θ and 29.5 °2Θ, with a step covering 0.02 °2Θ) and a time per step of 2 s. Computer analysis was performed using PANalytical X'Pert HighScore v2.0a. Background analysis was performed using a rolling average system (sonneveld and Visser, 1975). Following this peaks were located that had a minimum significance of 0.7, a minimum tip width of 0.1 °2Θ and a maximum tip width of 1 °2Θ. Peak height was then integrated assuming a Lorenzian distribution.

Pure crystalline griseofulvin or flavanone was subjected to the same methodology to produce a standard XRPD scan. A number of peaks were selected from each of these standards to produce a 100% crystalline value expressed as X-ray count/°2Θ these were then compared with the area under the peaks of the dispersions to produce percentage crystallinity values.

### Example 1

### The Effect of Different Minor Excipients on Stability

In this Example, griseofulvin was the first component to be dissolved. Specifically, either 6 g or 4 g of griseofulvin was dissolved in 240 ml of stirring acetone in a 500 ml conical flask.

To this was added 100 ml of distilled water. The mixture was then stirred for a minimum of 1 hour before the next component, PVP, was added in an amount as outlined in Table 1 below.

The secondary component was then dissolved in the stirring acetone/water solution as listed below (Table 1). Dispersions containing PAA required extra water in order to dissolve the PAA and PVP, and in total approximately 250 ml of water was used to create solutions containing all the constituents.

TABLE 1
(a list of the contents of various dispersions
and their amounts in grams)

| Dispersion | Mass of | Mass of | Name and mass of secondary |
|---|---|---|---|
| Name | Griseofulvin/g | PVP/g | component if present/g |
| Gris6PVP | 6 | 4 | None |
| Gris4PVP | 4 | 6 | None |
| Gris6Sucrose | 6 | 3 | Sucrose 1 |
| Gris4Sucrose | 4 | 4.5 | Sucrose 1.5 |
| Gris6PHPMA | 6 | 3 | PHPMA 1 |
| Gris4PHPMA | 4 | 4.5 | PHPMA 1.5 |
| Gris6PAA | 6 | 3 | PAA 1 |
| Gris4PAA | 4 | 4.5 | PAA 1.5 |

FIGS. 2 to 5 show the effects of different secondary components on the stability of Griseofulvin dispersions: As described in more detail above, accurately weighed griseofulvin was poured into 240 ml of stirring acetone contained within a 500 ml conical flask. 100 ml of distilled water was then added before the addition of the secondary polymer and then the PVP. Dispersions containing PAA required extra water in order to dissolve the PAA and PVP, and in total approximately 250 ml of water were used to create solutions containing all the constituents.

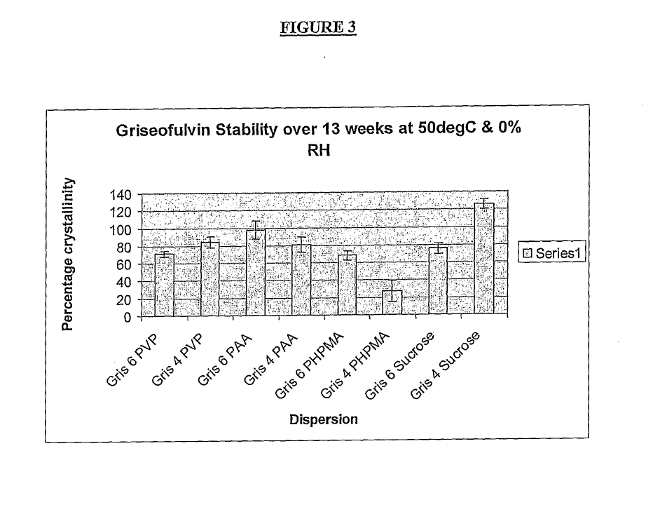

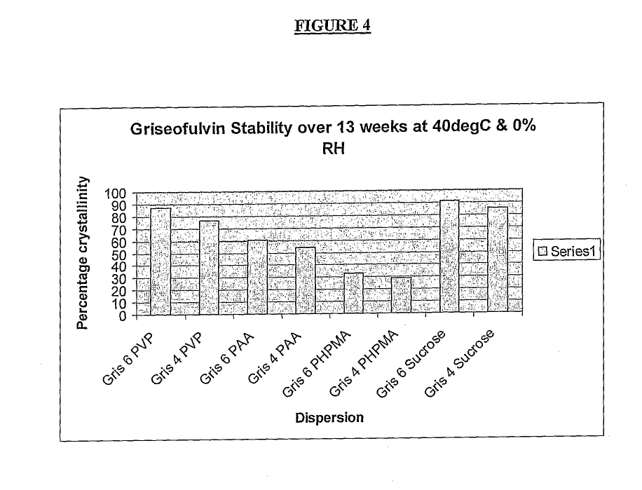

FIG. 2 shows the amorphous stability of the various dispersions after 13 weeks of storage at room temperature, 0% relative humidity (RH), with the addition of PHPMA clearly improving amorphous stability. FIG. 3 shows the same time point after storage at 50° C., 0% RH, and again the addition of PHPMA improves the amorphous stability of griseofulvin (data for Gris4suc suggests in excess of 100% crystallinity, most probably due to sucrose crystallisation). At 40° C., 0% RH, both PAA and PHPMA dispersions displayed improved amorphous stability over the Griseofulvin-PVP dispersions (FIG. 4).

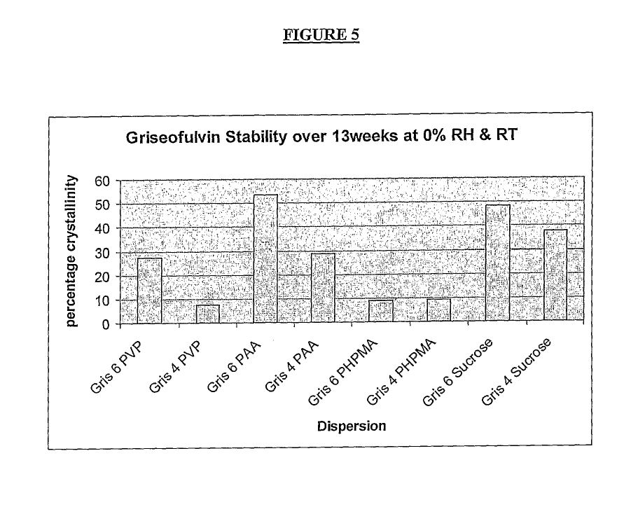

FIG. 5 shows the stability at room temperature, 0% RH and under these conditions only PHPMA was shown to improve amorphous stability.

### Example 2

### The Effect of Order of Addition on Spray Dried Griseofulvin Stability

A Griseofulvin-PVP dispersion was prepared as described in Example 1. This was followed by the preparation of Griseofulvin-PVP-PHPMA dispersions as follows:

### Griseofulvin-PVP-PHPMA

6 g of griseofulvin was dissolved in 240 ml of acetone. Once this had dissolved 100 ml of distilled water was added and then 3 g of PVP was dissolved in this co-solvent. Finally 1 g of PHPMA was added to the solution. The resultant solution was spray dried as discussed above.

### PHPMA-PVP-Griseofulvin

1 g of PHPMA was dissolved in 100 ml of distilled water. On dissolution 3 g of PVP was then added and allowed to dissolve. 6 g of griseofulvin was dissolved in 240 ml of acetone in a separate conical flask and this was then added to the PVP and PHPMA solution.

### Griseofulvin-PHPMA-PVP

6 g of griseofulvin was dissolved in 240 ml of acetone, then 100 ml of distilled water was added. Following this, 1 g of PHPMA and then 3 g of PVP were sequentially added and allowed to dissolve.

The samples were placed in a vacuum oven for 24 hours before being transferred to a desiccator and stored at 50° C., 0% RH.

Results from this experiment are shown in FIG. 6. In all situations the addition of PHPMA improved amorphous stability. The Griseofulvin-PHPMA-PVP system shows signs of crystallisation immediately after spray drying. This is probably due to non-ideal mixing prior to spray drying. Crystallisation is generally separated into two main sections, crystal nucleation and crystal growth, it was felt that subtracting the level of crystallites present at t=0 from the rest of the readings a usable rate of crystallisation could be observed hence a second line being drawn for the values of dispersion. Having done this the rate of crystallization was found to be similar for both the griseofulvin-PHPMA-PVP system and the PHPMA-Griseofulvin-PVP dispersion.

The Griseofulvin-PVP-PHPMA system showed the most improved amorphous stability. This is most likely due to two main effects; firstly, the Griseofulvin-PVP-PHPMA dispersion having the most ideally mixed composition, and secondly, that PHPMA is having the strongest bridging effect in this dispersion.

### Example 3

### The Effect of the Order of Mixing Using Different Proportions of Griseofulvin, PVP and PHPMA than Used in Example 2

Samples were prepared as in Example 2 except with the ratio Griseofulvin:PVP:PHPMA of 3:1:1. The stability is indicated in FIG. 7 where is can be seen that after 12 weeks at 84% RH the mixture of Gris+PHPMA then PVP was least stable, then Gris plus PVP then PHPMA and the most stable was PVP+PHPMA then Gris. As in Example 2, Gris then PHPMA then PVP was least stable, however on this occasion the most stable was when the two polymers were mixed first and then Gris added.

### Example 4

### The Effect of a Minor Excipient, PHPMA, on the Crystallization of Flavanone

Dispersions of Flavanone were prepared, using the same method as the Griseofulvin samples in Example 1 with the amounts of drug and polymer varied as shown below. XRPD analysis was performed by using a Flavanone peak at 22.5 °2Θ).

TABLE 2
(a list of the contents of various dispersions
and their amounts in grams)

| Sample number | Flavanone content | PHPMA content | PVP content |
|---|---|---|---|
| 1 | 4 g | 1.5 g | 4.5 g |
| 2 | 4 g | 3 g | 3 g |
| 3 | 4 g | 0 g | 6 g |

FIG. 8 shows the results from XRPD analysis of samples stored at room temperature under vacuum. The results indicate that whilst Flavanone appears to be near completely crystalline when dispersed with PVP, when dispersed with a combination of PHPMA and PVP only partial crystallinity is detected by XRPD.

### Example 5

### The Effect of Concentration Changes of a Minor Excipient, PHPMA, on the Crystallization of Griseofulvin

6 g of Griseofulvin was dissolved in 240 ml of acetone in a conical flask with the use of a magnetic stirrer. 200 ml of water was added to the flask. 2 g of PHPMA was added and the solution was left until the PHPMA had dissolved. Finally 2 g of PVP was added and allowed to dissolve. The solution was then spray dried and the spray drying process is described later.

Dispersion with 6 g of Griseofulvin, 1 g of PHPMA and 3 g of PVP was prepared using the same method and finally a dispersion with 6 g of Griseofulvin and 4 g of PVP was prepared. To prepare the solid dispersion of Griseofulvin alone, the Griseofulvin was dissolved in acetone alone and then spray dried as described previously. Storage took place at room temperature under vacuum

The results shown in FIG. 9 indicate that the level of crystallisation of Griseofulvin is again reduced by the addition of PHPMA and that by increasing the proportion of PHPMA with respect to the other constituents; one may be able to increase the level of amorphous stability still further.

### Example 6

### The Effect of Component Proportion on Composition Stability

Samples were prepared as in Example 5 with the ratios

### Gris:PHPMA:PVP

a) 2.5:0.25:2.25
b) 2.5:0.5:2.0
c) 2.5:0.75:1.75
d) 2.5:1.0:1.5
e) 2.5:1.25:1.25
f) 2.5:1.5:1

It can be seen from X-ray diffraction data in FIG. 10 that after storage for 12 weeks at 84% RH the was crystallinity in sample (a) some crystallisation in (b), but amorphous material in the other samples. This shows that the preferred proportions are for bridge to carrier ratio of 0.75:1.75 (i.e. 1:2.33 or 0.42:1) and for higher proportions of the bridging polymer (i.e. 0.5:1 to 1.5:1)

### Example 7

### Dissolution Data

Dispersions were prepared as described above containing Gris:PVP 1:1; Gris:PHPMA:PVP 2.5:1.25:1.25, and compared to Gris spray dried without polymer and crystalline Gris. Dissolution data were prepared by placing a small amount of sample in a hard gelatine capsule and following dissolution in pH6.8 buffer at 37 C (with added surfactant if dispersion was required), using a USP paddle method. Very small masses of material were used to maintain sink conditions; hence weighing errors did impact on 100% release values.

The results are shown in FIG. 11. Whilst the data for the dispersion containing PHPMA (a—in FIG. 11) were run with a slower stirring speed this formulation still showed the fastest dissolution response. The spray dried and crystalline material both resulted in slow dissolution and the dispersion of PVP and Gris had slower dissolution than did the sample with added PHPMA.

1-12. (canceled)

13. A method of forming a stable composition of a bioactive, comprising the steps of:
(i) providing a bioactive and a carrier wherein, if they alone are mixed, at least one of them has a tendency to crystallize,
(ii) mixing a bridging component with the bioactive and the carrier, wherein the bridging component forms hydrogen bonds with both the bioactive and the carrier, thereby forming a composition in which at least the bioactive has less of a tendency to crystallise than if the bridging component were not present,
wherein the bridging component is a hydrogen bond donor to both the bioactive and the carrier, and
wherein the carrier is polyvinylpyrrolidone and the bridging component is selected from polyhydroxypropylmethacrylate and polyacrylic acid.

14. The method according to claim 13, wherein the composition is prepared by first mixing the carrier and the bridging component and then admixing the bioactive.

15. The method according to claim 13, wherein the bioactive is griseofulvin,

16. The method according to claim 15, wherein the bridging component is polyhydroxypropylmethacrylate.

17. The method according to claim 13, wherein the composition components are combined by dissolving or dispersing them in at least one solvent which is evaporated after the components are sufficiently mixed.

18. The method according to claim 17, wherein said at least one solvent is evaporated by spray drying.

19. The method according to claim 13, wherein the composition components are combined by mechanical activation.

20. The method according to claim 13, wherein the composition comprises from 40 to 60% w/w of bioactive, and wherein the remainder comprises bridging component and carrier.

21. The method according to claim 13, wherein the ratio of carrier to bridging component is from 0:5:1 to 3:1.

22. The method according to claim 21, wherein the ratio of carrier to bridging component is from 0:5:1 to 1:5:1.

23. A composition obtainable by a method as claimed in claim 1.
